(19) United States
(12) Patent Application Publication
(10) Pub. No.: US 2016/0313316 A1
(43) Pub. Date: Oct. 27, 2016

(54) SURFACE ACOUSTIC WAVE SENSOR FOR INFLUENZA DETECTION

(71) Applicant: Agency for Science, Technology and Research, Singapore (SG)

(72) Inventors: Kui YAO, Singapore (SG); Chin Yaw TAN, Singapore (SG); Ying JIANG, Singapore (SG); Yi Fan CHEN, Singapore (SG); Sze Yu TAN, Singapore (SG); Lei ZHANG, Singapore (SG)

(21) Appl. No.: 15/103,477

(22) PCT Filed: Dec. 10, 2014
(86) PCT No.: PCT/SG2014/000588

§ 371 Date: Jun. 10, 2016

(30) Foreign Application Priority Data
Dec. 10, 2013 (SG) 201309151-7

(51) Int. Cl. G01N 33/543; G01N 29/02; G01N 33/569; G01N 29/32

(52) U.S. Cl. CPC G01N 33/54366; G01N 29/326; G01N 29/022; G01N 33/56983; G01N 2291/02466; G01N 2291/0256; G01N 2333/11

(57) ABSTRACT

An influenza detector for detecting a targeted influenza virus and a surface acoustic wave (SAW) sensor for Influenza A virus detection in liquid are provided. The influenza detector includes a liquid environment, the surface acoustic wave (SAW) sensor and an influenza specific binding agent such as an antibody. The agent is immobilized on a surface of the SAW sensor for selectively capturing an analyte for the targeted influenza virus. The SAW sensor is in contact with the liquid environment and includes a substrate comprising a piezo-electric material for producing a surface acoustic wave signal in response to an applied electric field and an insulative layer formed on top of the substrate and having a functionalized surface formed thereon for selectively immobilizing the influenza specific binding agent, the functionalized surface being in contact with the liquid environment. The surface acoustic wave signal produced by the SAW sensor changes in response to the analyte for the targeted influenza virus being present in the liquid environment and being captured by the influenza specific binding agent immobilized on the functionalized surface of the insulative layer of the SAW sensor.

### PRIORITY CLAIM

The present application claims priority to Singapore Patent Application No. 201309151-7, filed 10 Dec. 2013.

### FIELD OF THE INVENTION

The present invention relates to influenza detection. In particular, it relates to a surface acoustic wave sensor for influenza detection.

### BACKGROUND OF THE DISCLOSURE

Influenza is a common infectious respiratory disease, affecting people from rural areas as well as crowded urban areas. Its rampant spread in the form of new, deadly strains has become common, as has been notable from the recent outbreaks of bird flu and swine flu. Improved screening and diagnosis technologies at low cost for influenza virus are highly demanded by the medical industry, public welfare and society in general for effectively controlling the outbreak and spread of this disease.

Influenza is caused by three types of viruses, belonging to the virus family Orthomyxoviridae—Influenza A, B and C. Type A is responsible for the pandemics that break out every ten to forty years and affects about fifty per cent of the population, whereas, type B causes less severe, localized outbreaks. Type C, on the other hand, results in very mild symptoms and is rarer than the other two types, primarily causing mild symptoms in children. As Influenza A is the one that causes pandemics that widely spread among all groups of people across the world and threatens millions of human lives, low cost and portable tools for reliable Influenza A screening and diagnosis that could be used outside hospitals for a wide variety of point-of-care applications are desired.

Rapid Influenza Diagnostic Tests (RIDTs) are the currently the most widely used tool in diagnosing Influenza A as they are point-of-care kits which can be used without professional training. However, they are not selective, not reliable, not quantitative and hence often cannot lead to a conclusion without further lab testing confirmation. Although real time reverse transcriptase polymerase chain reaction (RT-PCR) is more selective and reliable than RIDTs and able to produce quantitative results, RT-PCR is time-consuming, more costly and requires professional training in handling, and is not available at the point-of-care, including at clinics.

Thus, what is needed are point-of-care Influenza A sensors that are portable and easy to use, and have the advantages of low cost, quantitative testing, fast delivery of results, improved sensitivity, selectivity and reliability. Furthermore, other desirable features and characteristics will become apparent from the subsequent detailed description and the appended claims, taken in conjunction with the accompanying drawings and this background of the disclosure.

### SUMMARY

According to the Detailed Description, an influenza detector for detecting a targeted influenza virus is provided. The influenza detector includes a liquid environment, a surface acoustic wave (SAW) sensor and a targeted bioactive influenza species. The targeted bioactive influenza species is immobilized on a surface of the SAW sensor for selectively capturing an analyte for the targeted influenza virus. The SAW sensor is in contact with the liquid environment and includes a substrate comprising a piezoelectric material for producing a surface acoustic wave signal in response to an applied electric field and an insulative layer formed on top of the substrate and having a functionalized surface formed thereon for selectively immobilizing the targeted bioactive influenza species, the functionalized surface being in contact with the liquid environment. The surface acoustic wave signal produced by the SAW sensor changes in response to the analyte for the targeted influenza virus being present in the liquid environment and being captured by the targeted bioactive influenza species immobilized on the functionalized surface of the insulative layer of the SAW sensor.

Additionally, in accordance with the detailed description, a surface acoustic wave (SAW) sensor for Influenza A virus detection in liquid is provided. The SAW sensor includes a piezoelectric material and an insulative layer formed on top of the piezoelectric material. The piezoelectric material produces an in-plane mode surface acoustic wave signal in response to an electric field and the insulative layer has a functionalized surface formed thereon for selectively immobilizing a targeted bioactive influenza species for capturing an analyte of the Influenza A virus in the liquid.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to illustrate various embodiments and to explain various principles and advantages in accordance with a present embodiment.

FIG. 1, comprising FIGS. 1A and 1B, illustrates a surface acoustic wave (SAW) sensor in accordance with a present embodiment, wherein FIG. 1A is a top planar view of a layout of electrodes on top of a substrate forming the delay line used in the SAW sensor and FIG. 1B is a scaled layout showing dimensions for a unit cell of an electrode-width single-phase unidirectional transducer (EWC/SPUDT) of the SAW sensor.

FIG. 2, comprising FIGS. 2A and 2B, depicts photographic views of SAW sensors in accordance with the present embodiment, wherein FIG. 2A illustrates a piezoelectric wafer with fabricated Love wave delay lines after film deposition and patterning processing and FIG. 2B illustrates one SAW delay line after dicing from the wafer of FIG. 2A.

FIG. 3, comprising FIGS. 3A and 3B, depicts photographic views the SAW sensor in accordance with the present embodiment, wherein FIG. 3A illustrates the SAW sensor mounted into a calibrated fixture with low-loss radio frequency (RF) probes and FIG. 3B illustrates an amplified photomicrograph of the RF probes in contact with the EWC/SPUDT electrodes of the SAW sensor.

FIG. 4, comprising FIGS. 4A and 4B, depicts a test chamber assembly with the SAW sensor in accordance with the present embodiment, wherein FIG. 4A illustrates a top planar view of the test chamber assembly and FIG. 4B illustrates a side cross-sectional view of the test chamber assembly.

FIG. 5 depicts a graph of measured S₂₁ phase versus time for a SAW delay line sensor with a functionalized SiO₂ surface in accordance with the present embodiment.

FIG. 6 depicts a graph of S₂₁ phase versus time under different conditions for a SAW sensor in accordance with the present embodiment.

FIG. 7 depicts a graph of S₂₁ phase change versus time for a surface functionalized SAW sensor in accordance with the present embodiment exposed to a H1N1 HA-Ag solution at various concentrations.

FIG. 8 depicts a schematic diagram of a single delay line phase shift measurement circuit for use with the SAW sensor in accordance with the present embodiment.

FIG. 9 depicts a schematic diagram of a phase shift measurement circuit with an additional reference line for thermal compensation for use with the SAW sensor in accordance with the present embodiment.

FIG. 10 depicts a block diagram for an electrical circuit system of a portable Influenza A detector in accordance with the present embodiment.

FIG. 11, comprising FIGS. 11A and 11B depicts bond rupturing using acoustic waves with the SAW sensor in accordance with the present embodiment, wherein FIG. 11A depicts using in-plane acoustic waves and FIG. 11B depicts using out-of-plane acoustic waves.

And FIG. 12 depicts bond rupturing using an acoustic transducer to remove non-specific bond for improving selectivity for the Influenza A SAW sensor in accordance with the present embodiment.

Skilled artisans will appreciate that elements in the figures are illustrated for simplicity and clarity and have not necessarily been depicted to scale. For example, the dimensions of some of the elements in the illustrations, block diagrams or flowcharts may be exaggerated in respect to other elements to help to improve understanding of the present embodiments.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. Herein, a portable, easy-to-use, low cost point-of-care (POC) Influenza A detector using a surface acoustic wave (SAW) sensor is presented in accordance with present embodiments having the advantages of quantitative testing, fast delivery of results, improved sensitivity, selectivity and reliability. SAW devices that are able to operate in the frequency range from MHz to GHz can be used for detecting Influenza A in accordance with the present embodiments and can be mass produced at low cost for POC applications. The technology covers a design of in-plane Love mode SAW delay lines and an effective surface functionalization process for immobilizing the targeted Influenza A antibody and antigen. In addition, the technology includes a method and design for removing non-specific bonding, a method and design for detecting the Influenza A antigen based on the phase shift of SAW sensors operating in liquid, and a design of electronic circuits and a system to realize a portable SAW Influenza A detector.

A present embodiment for the design and operation of devices that can detect Influenza A virus is provided which utilizes piezoelectric SAW sensors for detecting the Influenza A virus. The SAW sensors in accordance with the present embodiment include Love mode SAW delay lines on a ferroelectric-based piezoelectric substrate material with a waveguide layer on top. The surface of the waveguide layer is chemically functionalized prior to utilization in order to immobilize a targeted bioactive Influenza A species, preferably an Influenza A virus antibody. An analyte for Influenza A virus, preferably an antigen of the influenza virus, is captured at the functionalized surface in accordance with the present embodiment through the specific antigen-antibody interaction in a liquid environment. In this manner, the analyte can be detected by the change of the SAW signals within a radio frequency (RF) frequency range corresponding to the specific antigen-antibody interaction.

Among the commonly available substrate materials for SAW devices, ferroelectric crystal LiNbO₃ has a high dielectric permittivity. As a piezoelectric material, LiNbO₃ also has a high electromechanical coupling factor. The ferroelectric-based piezoelectric LiNbO₃ (41° YX) single crystal is preferably chosen as the substrate for producing SAW sensors in accordance with the present embodiment as 41° YX LiNbO₃ has the advantages of a high SAW velocity (˜4792 m/s), a large electromechanical coupling factor (k²: ˜17.2%), and a high dielectric constant (63). The high SAW velocity can facilitate the micro patterning and fabrication and the large k² means higher efficiency during the conversion between electrical and acoustic energy.

Although the SAW propagation of 41° YX LiNbO₃ is by a leaky SH wave mode (i.e., a shear wave or S-wave polarized in the horizontal plane), the addition of a waveguide layer on the LiNbO₃ substrate enables the generation of Love mode waves which are concentrated at the surface to produce surface sensitive devices. SiO₂ is preferably chosen as the waveguide materials as it has low shear velocity, which enables efficient coupling of SAW from the LiNbO₃ substrate into the SiO₂ layer. Furthermore, the SiO₂ layer is insulative and has a low degree of velocity variation with temperature change. The use of Love mode SAW using the 41° YX LiNbO₃ substrate with the SiO₂ waveguide layer also enables the resulting SAW sensors to be used for virus detection in a liquid medium or liquid environment with minimized mechanical energy loss, as Love mode in-plane propagation has a low mechanical damping effect in liquid. The high permittivity of the LiNbO₃ and the highly insulating property of the SiO₂ layer also reduce the electrical energy loss in the liquid medium at high frequencies.

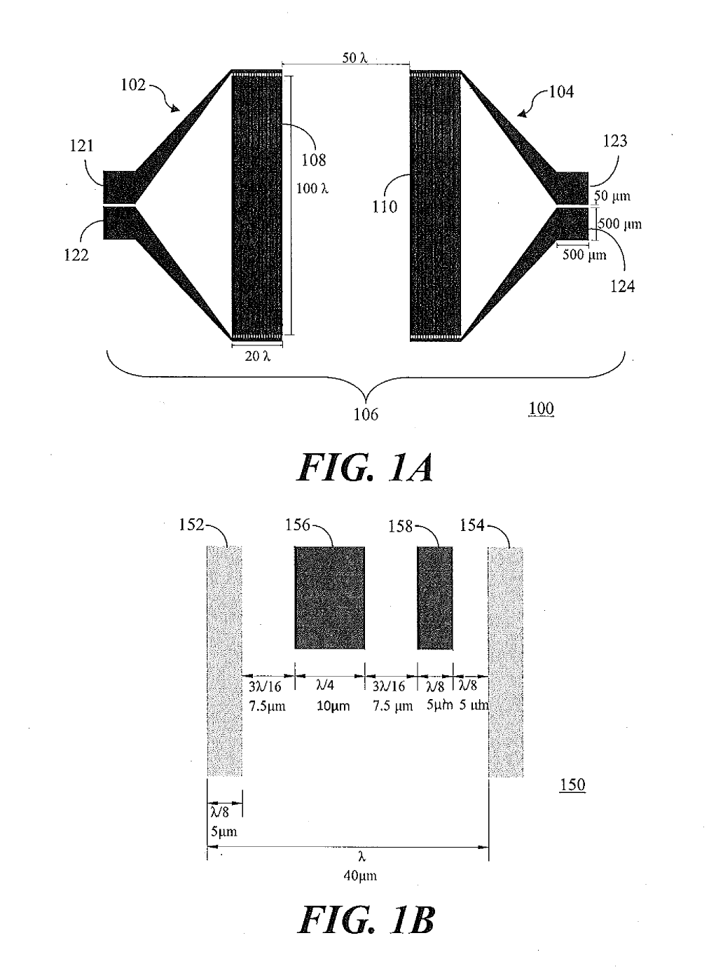

FIG. 1, comprising FIGS. 1A and 1B, illustrates a surface acoustic wave (SAW) sensor in accordance with a present embodiment. Referring to FIG. 1A, a top planar view 100 depicts a layout of electrodes 102, 104 on top of the LiNbO₃ substrate which form a two-port SAW delay line 106 used in the SAW sensor in accordance with the present embodiment. The two-port delay line 106 comprises a pair of electrode-width control/single-phase unidirectional transducers (EWC/SPUDTs) 108, 110. The EWC/SPUDT design has the advantage of single direction SAW propagation, unlike conventional bi-directional inter-digital transducers (IDTs). In addition, the EWC/SPUDT design minimizes the effect of multiple reflections from substrate edges during operation. Furthermore, the implementation of an absorber at edges of the substrate becomes not critical and, thus, not required.

FIG. 1B depicts a scaled layout 150 showing dimensions for a unit cell of the EWC/SPUDT of the SAW sensor. The minimum line and gap width is ⅛λ (where λ is the SAW wavelength), as shown in FIG. 1B. For a SAW sensor with nominal operating frequency at 120 MHz, a wavelength λ of 40 μm (based on SAW propagation velocity of the 41° YX LiNbO₃) and a minimal line and gap width of 5 μm can be conveniently realized with standard photolithographic patterning processes. Coupling-of-mode simulation was utilized for the SAW delay line 106 design.

The fabrication of the designed SAW delay line 106 guided by COM analysis was started with a 4-inch 41° YX LiNbO₃ wafer. An aluminum (Al) electrode with a thickness of 80 nm was deposited by an e-beam evaporation process and patterned by photolithography and standard wet Al etching. A 200 nm-thick gold (Au) layer was deposited by e-beam deposition and patterned by lift-off process at the locations of the electrode pads 102, 104 to increase the thickness of the electrode pads. A 2 μm-thick insulative SiO₂ layer was deposited by plasma-enhanced chemical vapor deposition (PECVD) and patterned through a standard photolithography followed by a reaction ion etching (RIE) process such that the electrode pads 121, 122, 123 and 124 are not covered by the SiO₂ layer.

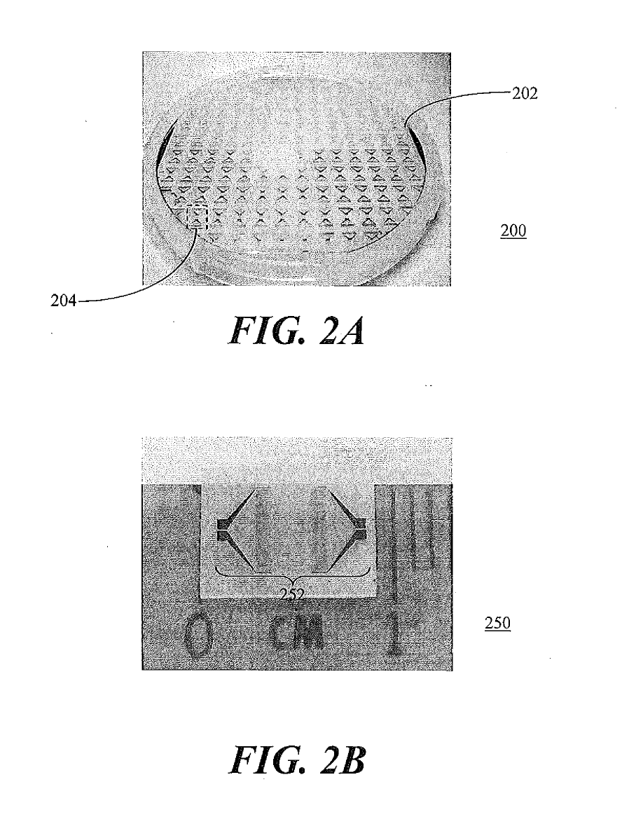

FIG. 2, comprising FIGS. 2A and 2B, depicts photographic views 200, 250 of SAW sensors in accordance with the present embodiment. Referring to FIG. 2A, a four-inch piezoelectric LiNbO₃ wafer 202 includes fabricated Love wave delay lines 204 after film deposition and patterning processing. The wafer 202 is then diced into individual SAW delay lines 252 as shown in FIG. 2B, each individual SAW delay line 252 measuring 9 mm by 6.5 mm.

Surface functionalization is then performed to create a bioactive SiO₂ surface on top of the SAW delay line. The process starts with cleaning the SAW substrate with SiO₂ in a hot piranha solution (concentrated H₂SO₄ and 30% H₂O₂ in 70:30 volume ratio at 85° C.) for fifteen minutes. After thoroughly rinsing the processed substrates in water, they are dried and transferred into an inert nitrogen glove box. In the glove box, the substrates are soaked in a solution of triethoxysilylbutylaldehyde (ALTES) in absolute ethanol (e.g., 0.457 M ALTES in ethanol) for two hours. The samples are then washed thoroughly with ethanol and dried at 110° C. for thirty minutes.

### EXAMPLE 1

Immobilization of H1N1 hemagglutinin (HA) antibodies (anti-HA) on the functionalized surface was carried out by soaking the substrates in a 16.5 μg/ml solution of the antibodies in 0.05 M phosphate buffered saline (PBS) overnight at room temperature on a shaker that operates at 75 rpm. Antibodies conjugated to the phycoerythrin (PE) fluorophore (anti-HA-PE) were immobilized for fluorescence microscopy analysis of the functionalized surface and an observed fluorescence emission indicated that anti-HA is successfully immobilized on the SiO₂ surface.

Antibodies without fluorophore conjugation (anti-HA) were also used for actual SAW sensor testing. Surfaces that would be characterized for fluorescence emission were kept in the dark to prevent bleaching of fluorophores under ambient lab light. Before following antigen deposition, the surfaces were passivated by soaking samples in 1M ethanolamine for one hour.

The ALTES/anti-HA-PE surfaces were then exposed to fluorescent HA antigen (HA-Ag) conjugated to the fluorophore fluorescein isothiocyanate (FITC). The substrates were soaked in a 100 ng/ml solution of the fluorescent antigen (HA-FITC) and shaken at 75 rpm overnight at room temperature. The functionalized surfaces were characterized with confocal fluorescence microscopy and the fluorescence emission indicated that HA-Ag was successfully immobilized on the SiO₂/ALTES surface. The HA-Ag without FITC conjugation was also used in actual SAW sensor testing.

A SAW sensor Influenza A detector refers to the LiNbO₃/SiO₂ SAW delay line with the chemically functionalized SiO₂ surface, such as with ALTES, and the targeted bioactive Influenza A species, preferably the Influenza A virus antibodies, immobilized on the functionalized SiO₂ surface.

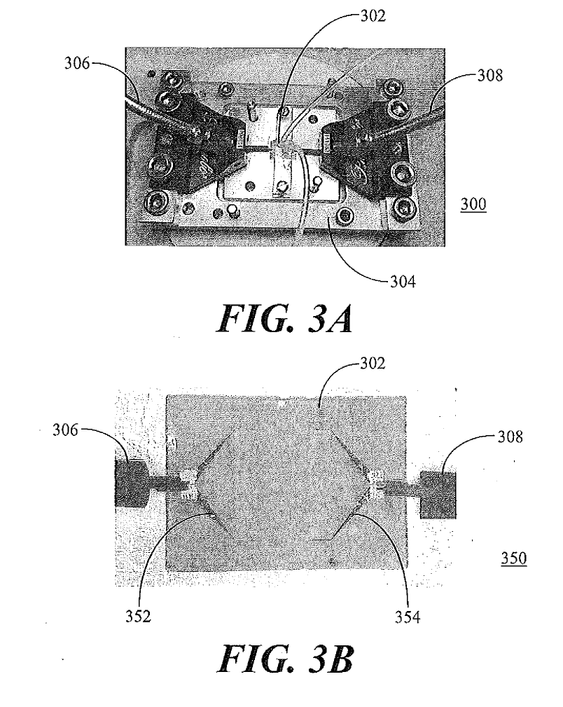

FIG. 3, comprising FIGS. 3A and 3B, depicts photographic views 300, 350 of the SAW sensor in accordance with the present embodiment. FIG. 3A illustrates a SAW sensor 302 mounted into a calibrated fixture 304 with low-loss radio frequency (RF) probes 306. FIG. 3B illustrates an amplified photomicrograph 350 of the RF probes 306 in contact with the EWC/SPUDT electrodes 352, 354 of the SAW sensor 302.

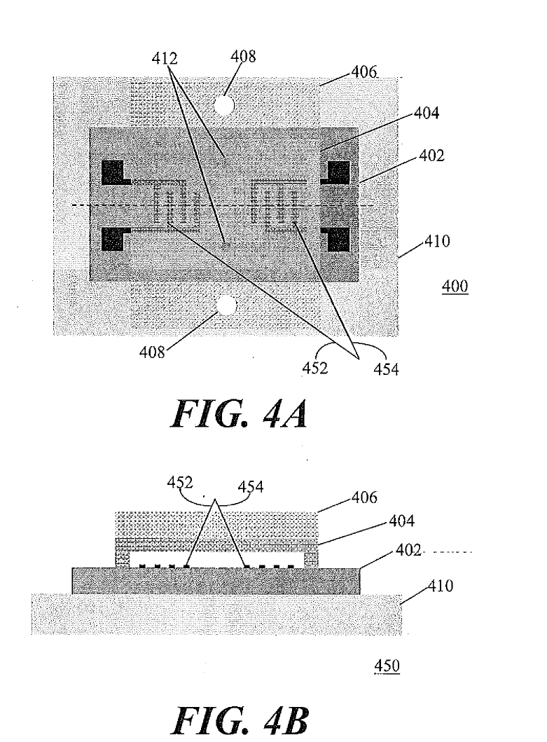

FIG. 4A illustrates a top planar view 400 of a test chamber assembly with a SAW sensor 402 in accordance with the present embodiment. FIG. 4B illustrates a side cross-sectional view 452 of the test chamber assembly. A cover 404 of the chamber is made of polydimethylsiloxane (PDMS) or silicone, which is a stable sealing material. An acrylic plate 406 is placed over the PDMS cover 404 and secured using screws through mounting holes 408 to a base plate 410 to ensure no leakage from the interface between the PDMS cover 404 and the sensor chip 402. As shown in FIGS. 3 and 4, there are two holes 408 at the ends of the acrylic plate 406 for the screws. Near the centre of the acrylic plate 406, there are two holes 412 which allow the input/output fluid tubing to pass through.

The three layers comprising the SAW sensor 402, the PDMS cover 404, and the acrylic plate 406 are aligned so the EWC/SPUDT electrodes 452, 454 are enclosed by the PDMS cover 404 but without direct contact with the walls of the PDMS cover 404. The phase of the S₂₁ S-parameter was measured using a vector network analyzer. A chamber made from the PDMS (silicone) cover 404 and the acrylic plate 406 ensures no fluid leakage from the SAW sensor 402 when a liquid containing an analyte for Influenza A virus is pumped through the fluid tubing to provide a liquid environment in contact with the SAW sensor 402.

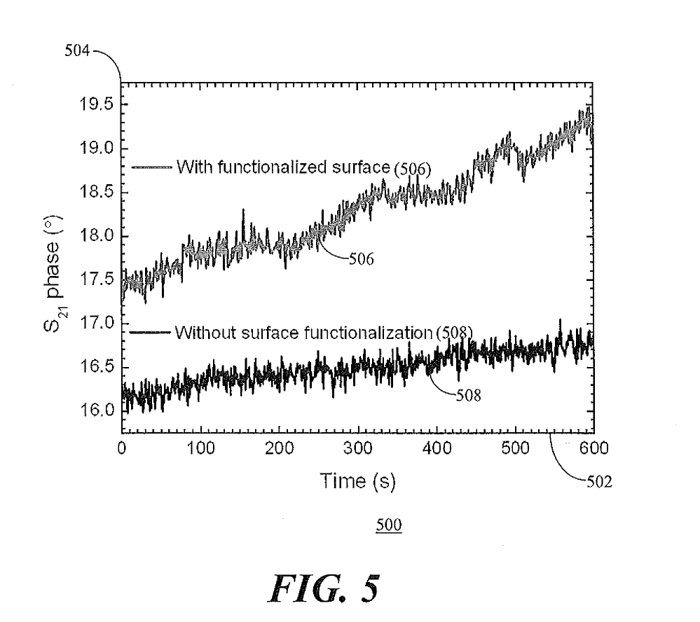

Referring to FIG. 5, a graph 500 of measured S₂₁ phase (along a y-axis 504) versus time (along a x-axis 502) for a SAW delay line sensor with a functionalized SiO₂ surface for capturing a H1N1 HA antigen (HA-Ag) in accordance with the present embodiment plotted along a trace 506 and another SAW delay line sensor without functionalizing the SiO₂ surface plotted along a trace 508 to act as a control. HA-Ag is an analyte for an Influenza A virus. Prior to measurement of data, a PBS solution was flowed through the chamber for 20 minutes. There is a significant increase of about 2.0° in the S₂₁ phase change of the SAW sensor with the surface functionalization at ten minutes as compared with the phase change of the control SAW sample of only about 0.6°. Moreover, the phase change of the SAW sensor is also significantly higher than the measurement noise of less than ±0.1°. The S₂₁ phase of the control SAW sensor of about 0.6° is probably due to the HA-Ag molecules nonspecifically interacting with the SiO₂ surface of the SAW control without surface functionalization. This result shows that the SAW sensor with the functionalized surface is able to clearly detect the presence of HA-Ag molecules.

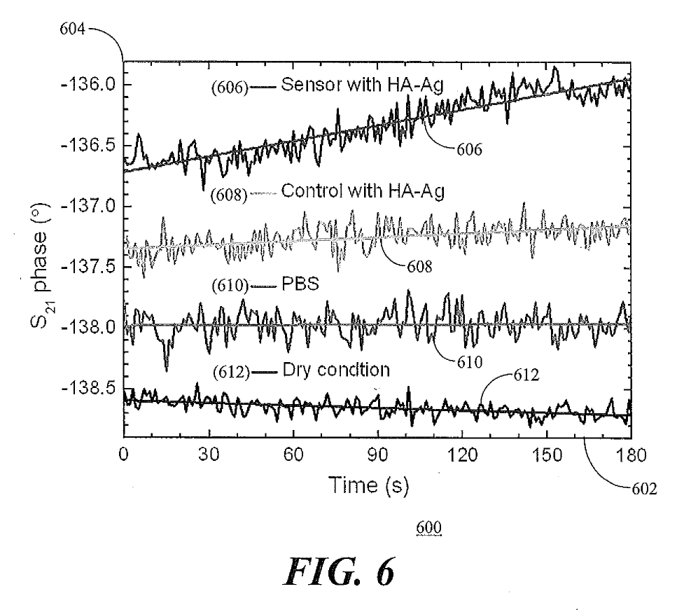

Referring to FIG. 6, a graph 600 of S₂₁ phase (plotted along a y-axis 604) versus time (plotted along a x-axis 602) under different conditions for a SAW sensor in accordance with the present embodiment is depicted. Note that an offset constant is added to the data for different conditions to enable the display of multiple datasets on one graph 600.

To further evaluate the noise level and possible drift errors of the measurement, linear fitting was applied to data measured at the following different conditions: (a) a surface-functionalized SAW sensor with a H1N1 HA-Ag solution having a 100 ng/ml concentration (trace 606 in the graph 600); (b) a control of the SAW delay line sample without surface functionalization in the H1N1 HA-Ag solution (trace 608); (c) with PBS (trace 610); and (d) when the SAW sensor was dry (trace 612). The data of the calculated gradient of S₂₁ phase versus time and root mean square error (root MSE) are also provided in Table 1. The gradient reflects the drift errors or actual measurement change, depending on measurement conditions, and the root MSE is an indication of the measurement noises. From the calculated root MSE, the noise of the measurement is less than ±0.2°. Also, the possible drift error, calculated from the gradient is −6.5×10⁻⁴°/s under dry conditions, and is 1.1×10⁻⁴°/s under wet condition (PBS).

The gradient for the measurement with the SAW control in the HA-Ag solution is about 9.7×10⁻⁴°/s and the gradient for the measurement with the surface functionalized SAW sensor in the HA-Ag solution is about 4.3×10⁻³°/s, which is significantly larger than that of the control phase change and drift error. This result clearly indicates the viability of the SAW sensor with the surface functionalization for detecting H1N1 HA-Ag.

TABLE 1

| | Root MSE/noise | Gradient | Phase change |
|---|---|---|---|
| Condition | (°) | (10⁻⁴ °/s) | (°) |
| Dry | 0.06 | −6.5 | −0.39 |
| PBS | 0.11 | 1.1 | 0.07 |
| Control SAW delay | 0.10 | 9.7 | 0.58 |
| line with HA-Ag | | | |
| (conc. 100 ng/ml) | | | |
| solution | | | |
| Surface functionalized | 0.10 | 43 | 2.60 |
| SAW sensor | | | |
| with HA-Ag | | | |
| (conc. 100 ng/ml) | | | |
| solution | | | |

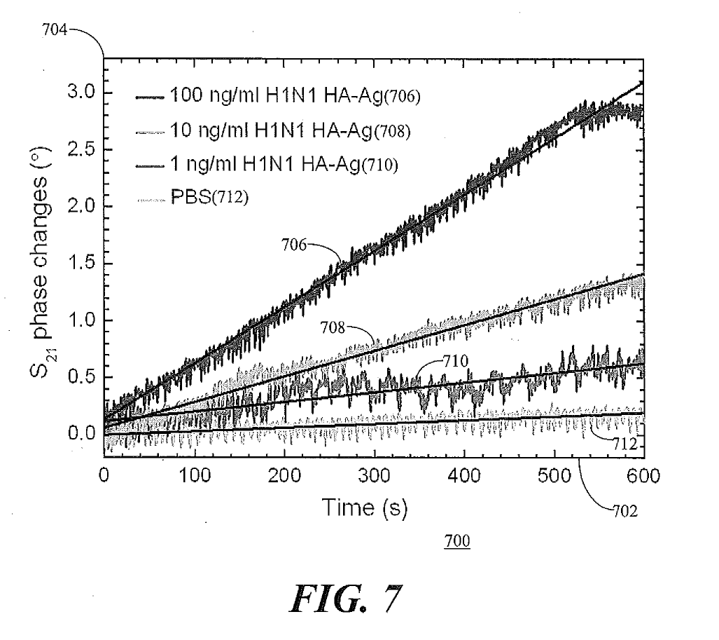

FIG. 7 depicts a graph 700 of S₂₁ phase change (plotted along a y-axis 704) versus time (plotted along a x-axis 702) for a surface functionalized SAW sensor in accordance with the present embodiment exposed to a H1N1 HA-Ag solution at various concentrations, Traces 706, 708, 710 and 712 correspond to H1N1 HA-Ag solutions at concentrations of 100 ng/ml, 10 ng/ml, 1 ng/ml and zero (i.e., PBS), respectively. From the graph 700, those skilled in the art will realize that the SAW sensor is able to quantitatively detect H1N1 HA-Ag with a sensitivity resolution at a concentration of 1 ng/ml and even lower. The S₂₁ phase change for 1 ng/ml HA-Ag solution over 10 minutes is 0.5° (trace 710), which is substantially higher than the baseline drift of 0.2° and noise of 0.1° without any compensation design and under normal operation environment at room temperature. The solution flow rate for the set of measurements in the graph 700 and in Table 2 are reduced from the previous 0.2 ml/min to 0.02 ml/min, which means the sample/specimen volume required was further reduced. In addition, the RF measurement bandwidth was narrowed to reduce the noise in the graphs 500 and 600.

TABLE 2

| | Root MSE/noise | Gradient | Phase change |
|---|---|---|---|
| Concentrations | (°) | (10⁻⁴°/s) | (°) |
| PBS | 0.06 | 3.3 | 0.20 |
| HA-Ag solution, 1 ng/ml | 0.10 | 8.5 | 0.51 |
| HA-Ag solution, | 0.07 | 23 | 1.38 |
| 10 ng/ml | | | |
| HA-Ag solution, | 0.09 | 49 | 2.94 |
| 100 ng/ml | | | |

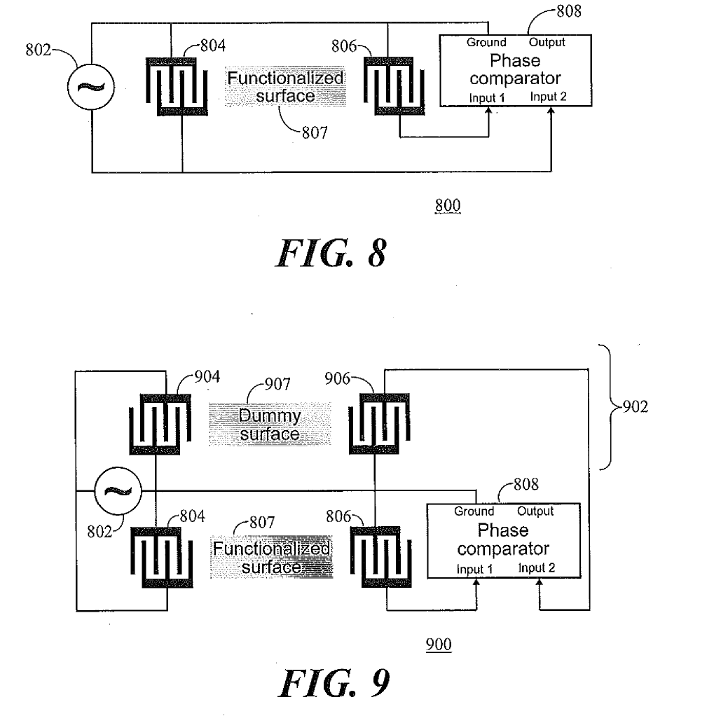

FIG. 8 depicts a schematic diagram 800 of a single delay line phase shift measurement circuit for use with the SAW sensor in accordance with the present embodiment. The electrical circuit and system are designed to build a portable detector for Influenza A virus detection based on the responsive S₂₁ phase shift of the SAW sensors as demonstrated. A signal source 802 is required to drive the input SAW transducer 804. The output SAW transducer 806, separated from the input SAW transducer 804 by the functionalized surface 807, is connected to a phase comparator 808. The phase comparator 808 is also connected directly to the signal source 802. The phase comparator 808 is an integrated circuit that is commercially available.

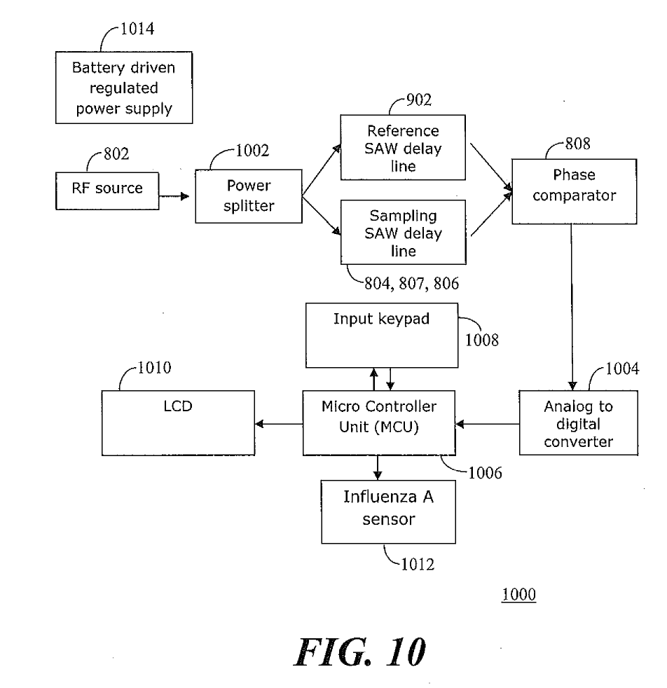

The thermal stability of the measurement circuit based on the phase shift measurement can also be improved using a reference delay line as shown in FIG. 9. FIG. 9 depicts a schematic diagram 900 of the phase shift measurement circuit with an additional reference line 902 for thermal compensation for use with the SAW sensor in accordance with the present embodiment. The SAW delay line control as described above without the surface functionalization can be used as the dummy. This SAW delay line includes an input SAW transducer 904 and an output SAW transducer 906 separated by a dummy surface 907. The input SAW transducer 904 is connected to the signal source 802 and the output SAW transducer 906 is connected to the phase comparator 808.

The main advantage of the phase shift based measurement method in accordance with the present embodiment is better stability without the problem of amplifier instability and multi-modal frequency hopping of the delay line. Cost of the phase shift-based measurement circuit is also low although higher than a delay line oscillator based method as a low noise, high phase stability signal source 802 is required.

In addition to the measurement electronic circuit, the implementation of a SAW Influenza A sensor also requires an analog to digital conversion circuit for converting the output analog signal to a digital signal for readout on a LCD display with a programmable integrated microprocessor or on a laptop computer.

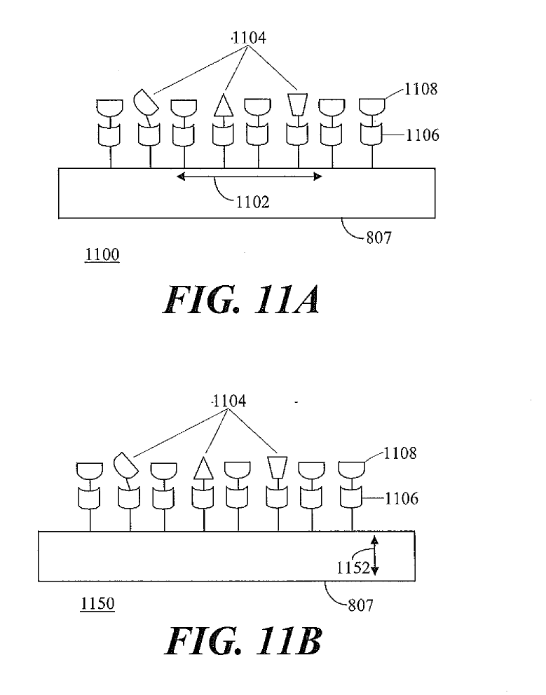

Referring to FIG. 10, a block diagram 1000 for an electrical circuit system of a portable Influenza A detector in accordance with the present embodiment. The RF signal source 802 is connected to a power splitter 1002 which applies the signal source to both the reference SAW delay line 902 and the sampling SAW delay line 804, 807, 806. Both delay lines provide their signal to the phase comparator 808 (as shown in more detail in FIG. 9) which outputs the analog comparator signal to an analog to digital converter 1004. The analog to digital converter 1004 provides a digital signal corresponding to the analog comparator signal to a microcontroller unit (MCU) 1006 which is coupled to a user interface including, for example, an input keypad 1008, a liquid crystal display (LCD) 1010 and an Influenza A sensor 1012, the Influenza A sensor 1012 interpreting the data from the MCU 1006 to determine whether Influenza A analyte is present. A battery driven regulated power supply 1014 is used to provide power to the system thereby providing a portable, low cost point-of-care Influenza A detector.

### EXAMPLE 2

Instead of immobilization of H1N1 anti-HA as a bioactive Influenza A species to selectively detect the corresponding HA-Ag as the analyte for the H1N1 virus as in Example 1, another bioactive Influenza A species, H1N1 nucleoprotein antibodies (anti-NP), was immobilized on the functionalized surface of the SAW delay line to detect the corresponding H1N1 nucleoprotein antigen (NP-Ag), as the analyte for the H1N1 virus in this Example 2.

Surface functionalization was conducted to make a bioactive SiO₂ surface on top of the SAW delay line 804, 807, 806. The process starts with cleaning the SAW substrate with SiO₂ in a hot piranha solution (concentrated H₂SO₄ and 30% H₂O₂ in 70:30 volume ratio at 85° C.) for fifteen minutes then thoroughly rinsing the substrate in water, drying it and transferring it into an inert nitrogen glove box. In the glove box, the substrate(s) is soaked in a solution of triethoxysilylbutylaldehyde (ALTES) in absolute ethanol (e.g. 0.457 M ALTES in ethanol) for two hours. The samples are then washed thoroughly with ethanol and dried at 110° C. for thirty minutes.

To make a bioactive SiO₂ surface on top of the SAW delay line, immobilization of anti-NP was carried out by soaking the SAW substrates in a 16.5 μg/ml solution of the anti-NP in 0.05 M PBS buffers overnight at room temperature on a shaker that operates at 75 rpm. Verification for the immobilization of anti-NP was carried out by soaking the surface in 1 mg/ml FITC in dimethylsulfoxide (DMSO). The presence of anti-NP bonded FITC creates a fluorescent surface. Before antigen deposition, passivation was carried out by soaking samples in 1 M ethanolamine for three hours.

The ALTES/anti-NP surfaces can selectively capture fluorescent NP antigen conjugated to the Alexa Fluor 488 (NP-Alexa). Prior to conjugation, 0.4 ml each of 4 μg/ml solutions of NP and Alexa Fluor 488 were mixed, and the solution was shaken at 75 rpm overnight at room temperature, achieving a final concentration of 2 μg/ml for each solution. The NP-Alexa conjugate solution was further diluted to 100 ng/ml for surface immobilization. Fluorescence microscopy images for ALTES control, ALTES/anti-NP and ALTES/anti-NP/NP-Alexa surfaces confirmed that anti-NP and NP-Ag were successfully immobilized at the surface of the SiO₂ on the SAW substrates. For subsequent SAW sensor testing, anti-NP and NP-Ag without conjugation to fluorescent agents were used.

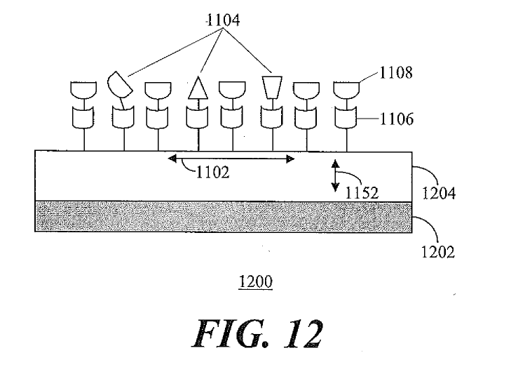

Besides the bond formations between the chemical groups on the functionalized surface (such as those from ALTES in Examples 1 and 2) and antibodies, and the bond formations between Influenza A antigens and antibodies, other unintended non-specific non-covalent bonds may also be formed in the solutions, which may unfavorably affect the selectivity for the targeted Influenza A antibodies and antigens. In Examples 1 and 2, during or after the processing for immobilizing the Influenza A antibodies and antigens, but before SAW sensor testing, in accordance with the present embodiment, acoustic waves in the SAW substrate are introduced to rupture the non-specific non-covalent bonds, which are usually weaker than the targeted bonds, such as between antibodies and antigens, for improving the selectivity and sensitivity of the SAW Influenza A sensors. Referring to FIG. 11, including FIGS. 11A and 11B, this bond rupturing using acoustic waves with the SAW sensor in accordance with the present embodiment is depicted in diagrams 1100 and 1150, respectively. FIG. 11A depicts using in-plane acoustic waves 1102 for bond rupturing of non-specific bonds 1104 between antibodies 1108 and antigens 1106 on the functionalized surface 807 of the SAW sensor. The in-plane acoustic waves 1102 are the same as the Love wave delay line as in Examples 1 and 2, or other shear horizontal waves. The in-plane acoustic waves 1102 will produce shear stress to the non-specific non-covalent bonds 1104 which will eventually rupture the bonds 1104 when the shear acoustic waves are strong enough.

FIG. 11B depicts using out-of-plane acoustic waves 1152 for bond rupturing where the amplitude of the out-of-plane acoustic waves 1152 are perpendicular to the functionalized surface 807 of the SAW sensor. The out-of-plane acoustic waves 1152 will produce tensile stress to the non-specific bonds 1104 and will rupture the bonds if the acoustic wave is strong enough.

Many of the non-specific non-covalent bonds are weak and bond rupture forces could be in the range of a few pica Newtons (pN). A surface acoustic wave at a high frequency can produce the force well above pN level, which can be enough to rupture some specific non-covalent antibody-antigen bonds 1104. Thus, in accordance with the present embodiment, appropriately adjusting the SAW intensity can effectively rupture the non-specific non-covalent bonds 1104 and advantageously improve the selectivity and sensitivity of the Influenza A SAW sensors.

Theoretical calculations have shown that for a mass of 56 kDa (i.e., approximately a mass of Influenza A NP), which is equivalent to m=9.63×10⁻²³ kg, and an acoustic wave with out-of-plane amplitude of A₀=3 nm, the inertia force applied to the Influenza NP can be calculated by F=mA₀(2πf)², which is 11.4 pN at 1 GHz. This is of the same order for the tensile rupture force of many non-covalent bonds.

For bond rupture using shear waves, the force required to rupture a bond is usually even lower than that of the tensile rupture force. The ultimate shear force can be estimated by using Von Mises yield criteria which is 0.577 times ultimate tensile strength. In addition, if elevated temperatures can be introduced simultaneously, the force required for bond rupture can be further reduced. For the removal of non-specific bonding, the acoustic force should be maintained below the rupture force of the antigen-antibody bond.

Referring to FIG. 12, a diagram 1200 depicts bond rupturing using an ultrasonic acoustic transducer 1202 to remove the non-specific bonds 1104 for improving selectivity for the Influenza A SAW sensor 1204 in accordance with the present embodiment. The ultrasonic transducer 1202 is in contact with the SAW sensor 1204 and can be either an actuator working in shear or thickness mode producing horizontal vibrations 1102 or vertical vibrations 1152 to the SAW sensor 1204 or a typical ultrasound transducer which produces strong acoustic waves to be transmitted to the SAW sensor 1204.

Thus, it can be seen that a Love wave SAW design using ferroelectric-based piezoelectric material is utilized in the SAW Influenza A sensors working in a liquid environment in accordance with present embodiments. The losses into the bulk of the piezoelectric material or into the liquid above the sensor surface can be minimized, and thus these sensors are technically suitable for operation in the liquid environment for Influenza A detection with high sensitivity. In accordance with the present embodiments, the Influenza A SAW sensors are working in liquid environment in a liquid chamber with inlet and outlet fluid tubing to pass through. The SAW sensors' surfaces are effectively functionalized for immobilizing the corresponding Influenza A virus antibody to specifically bind the Influenza A antigen species in the liquid environment for realizing Influenza A detection. The phase shift of the S₂₁ S-parameter is measured within the RF frequency range to quantitatively determine the Influenza A antigen. In addition, in accordance with the present embodiments, an electrical circuit 1000 and system are disclosed to realize a portable Influenza A detector using the SAW sensor for point-of-care applications.

Those skilled in the art will realize that surfaces that possess functional groups with affinity for virus antibodies and other biomolecules have traditionally been created through the deposition, on silica of amino silanes (e.g. 3-aminopropyltriethoxysilane, APTES) or epoxysilanes (e.g. 3-glycidopropyltrimethoxysilane, GOPTS) followed by coupling with glutaraldehyde. In the case of APTES, amidization with succinic anhydride is also capable of activating the functionalized surface towards biomolecule adhesion. These methods of surface preparation involve multiple steps of activation and atmospheric disturbances that could potentially disrupt or deactivate the chemically sensitive surfaces. APTES is well known to undergo a variety of undesired interactions with silica surfaces upon exposure to slight atmospheric variations, prohibiting the formation of a functional surface. In accordance with the present embodiments, ALTES is advantageously deposited on the sensor surfaces in a single step. The functionalized surfaces so formed subsequently enable the effective adhesion of H1N1 virus antibodies (hemaglutinin and nucleoprotein), which are then active for specifically capturing their respective antigens to realize robust influenza A detection.

In addition, in accordance with the present embodiments, mechanical bond rupture methods realized through the use of the acoustic wave in the SAW sensors and/or introduction of another electromechanical transducer have been proposed to remove unintended nonspecific bonds thereby further improving the selectivity for immobilizing the targeted antibody and the targeted antigen analytes.

Thus it can be seen that Influenza A detectors using SAW sensors in accordance with the present embodiments have the advantages of portability, ease of use to enable point-of-care applications, low cost, quantitative testing, fast delivery of results, and improved sensitivity, selectivity and reliability. While exemplary embodiments have been presented in the foregoing detailed description of the invention, it should be appreciated that a vast number of variations exist.

It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of elements and method of operation described in an exemplary embodiment without departing from the scope of the invention as set forth in the appended claims.

1. An influenza detector for detecting a targeted influenza virus, the influenza detector comprising:
a liquid environment;
a surface acoustic wave (SAW) sensor in contact with the liquid environment; and
a targeted bioactive influenza species immobilized on a surface of the SAW sensor for selectively capturing an analyte for the targeted influenza virus,
wherein the SAW sensor comprises:
a substrate comprising a piezoelectric material for producing a surface acoustic wave signal in response to an applied electric field; and
an insulative layer formed on top of the substrate and having a functionalized surface formed thereon for selectively immobilizing the targeted bioactive influenza species, the functionalized surface being in contact with the liquid environment, and
wherein the surface acoustic wave signal produced by the SAW sensor changes in response to the analyte for the targeted influenza virus being present in the liquid environment and being captured by the targeted bioactive influenza species immobilized on the functionalized surface of the insulative layer of the SAW sensor.

2. (canceled)

3. The influenza detector in accordance with claim 1 wherein the piezoelectric material is a ferroelectric material with a dielectric constant greater than fifty at a working frequency of the surface acoustic wave signal.

4. The influenza detector in accordance with claim 1 wherein the SAW sensor further comprises a two-port delay line formed on the substrate into a pair of electrode-width single-phase unidirectional transducers, and wherein the surface acoustic wave signal comprises an in-plane mode surface acoustic wave signal which changes in response to the presence of the analyte for Influenza A virus in the liquid environment, the change comprising a phase shift of a radio frequency (RF) range S₂₁ S-parameter.

5. The influenza detector in accordance with claim 4 wherein a minimum width of electrodes of each of the pair of electrode-width single-phase unidirectional transducers is ⅛ of a wavelength of the surface acoustic wave signal.

6. The influenza detector in accordance with claim 4 wherein the in-plane mode surface acoustic wave signal comprises a Love mode wave signal, and wherein the insulative layer formed on the top of the substrate functions as a waveguide.

7-11. (canceled)

12. The influenza detector in accordance with claim 1 further comprising a liquid chamber for containing the liquid environment, the liquid chamber coupled to inlet and outlet fluid tubing for passing a supply of a liquid sample through the liquid environment, the liquid sample possibly including the analyte for the targeted influenza virus.

13. The influenza detector in accordance with claim 12 wherein the liquid chamber includes the functionalized surface of the insulative layer and a PDMS (polydimethylsiloxane) cover.

14. The influenza detector in accordance with claim 1 wherein mechanical energy is applied to the SAW sensor for mechanically rupturing nonspecific bonds with the functionalized surface thereby improving sensor selectivity of the SAW sensor, and wherein the mechanical energy is provided to the functionalized surface by the surface acoustic wave signal excited by applying the electric field to the SAW sensor.

15. (canceled)

16. The influenza detector in accordance with claim 1 wherein mechanical energy is applied to the SAW sensor for mechanically rupturing nonspecific bonds with the functionalized surface thereby improving sensor selectivity of the SAW sensor, and wherein the mechanical energy is provided to the liquid environment by an electromechanical transducer physically contacting the SAW sensor.

17. (canceled)

18. The influenza detector in accordance with claim 1 further comprising an electrical circuit coupled to the SAW sensor for applying the electric field to the piezoelectric material of the substrate.

19. The influenza detector in accordance with claim 18 wherein the electrical circuit comprises a phase shift measurement circuit with an additional reference line for thermal compensation.

20. A surface acoustic wave (SAW) sensor for Influenza A virus detection in liquid, the SAW sensor comprising:
a piezoelectric material for producing an in-plane mode surface acoustic wave signal in response to an electric field; and
an insulative layer formed on top of the piezoelectric material and having a functionalized surface formed thereon for selectively immobilizing a targeted bioactive influenza species for capturing an analyte for the Influenza A virus in the liquid.

21. The SAW sensor for Influenza A virus detection in liquid in accordance with claim 20 wherein the insulative layer formed on top of the piezoelectric material has a silane-functionalized surface formed thereon for selectively immobilizing a targeted bioactive influenza species for capturing HA antigen as an analyte for the Influenza A virus in the liquid.

22. The SAW sensor for Influenza A detection in liquid in accordance with claim 20 wherein the piezoelectric material comprises a ferroelectric material with a dielectric constant greater than fifty at a working frequency of the SAW sensor.

23. (canceled)

24. The SAW sensor for Influenza A detection in liquid in accordance with claim 20 further comprising:
a substrate comprising the piezoelectric material; and
a two-port delay line formed on the substrate into a pair of electrode-width single-phase unidirectional transducers, wherein a minimum electrode width of the unidirectional transducers is ⅛ of a wavelength of the surface acoustic wave signal and a gap between the pair of unidirectional transducers is ⅛ of a wavelength of the surface acoustic wave signal.

25-27. (canceled)

28. The SAW sensor for Influenza A detection in liquid in accordance with claim 21 wherein the in-plane mode surface acoustic wave comprises a Love mode wave and the insulative layer formed on the top of the piezoelectric material functions as a waveguide.

29. The SAW sensor for Influenza A detection in liquid in accordance with claim 20 wherein mechanical energy is applied to the functionalized surface for mechanically rupturing nonspecific bonds to improve sensor selectivity of the SAW sensor.

30. The SAW sensor for Influenza A detection in liquid in accordance with claim 29 wherein the mechanical energy is provided through the surface acoustic wave signal excited in the SAW sensor.

31. The SAW sensor for Influenza A detection in liquid in accordance with claim 29 wherein the mechanical energy is provided through use of an electromechanical transducer physically contacting the SAW sensor.

32. The SAW sensor for Influenza A virus detection in liquid in accordance with claim 21 wherein silane molecules of the silane-functionalized surface are triethoxysilylbutylaldehyde (ALTES).
